(19) United States
(12) Patent Application Publication
(10) Pub. No.: US 2011/0212032 A1
(43) Pub. Date: Sep. 1, 2011

(54) POLYMER PARTICLES FOR NIR/MR BIMODAL MOLECULAR IMAGING AND METHOD FOR PREPARING THE SAME

(75) Inventors: Bong Hyun Chung, Daejeon (KR); Yong Taik LIm, Daejeon (KR); Jung Hyun Han, Chungcheongnam-do (KR)

(73) Assignee: Korea Research Institute of Bioscience and Biotechnology, Daejeon (KR)

(21) Appl. No.: 12/672,185

(22) PCT Filed: Aug. 20, 2008
(86) PCT No.: PCT/KR08/04820

§ 371 Date: Mar. 17, 2010

(30) Foreign Application Priority Data
Aug. 29, 2007 (KR) 10-2007-0086850

(51) Int. Cl. A61K 49/12; C12Q 1/02

(52) U.S. Cl. 424/9.322; 435/29

(57) ABSTRACT

The present invention relates to polymer particles showing magnetic properties and fluorescent properties simultaneously and a preparation method thereof More specifically, relates to polymer particles for near infrared (NIR)/magnetic resonance (MR) bimodal molecular imaging, contain a magnetic nanomaterial and an NIR fluorescent material, and a preparation method thereof The disclosed polymer particles show magnetic properties and fluorescent properties simultaneously, and thus are useful as contrast agents for NIR/MR bimodal molecular imaging.

### TECHNICAL FIELD

The present invention relates to polymer particles showing magnetic properties and fluorescent properties simultaneously and a preparation method thereof, and more particularly to polymer particles for near infrared (NIR)/magnetic resonance (MR) bimodal molecular imaging, containing a magnetic nanomaterial and an NIR fluorescent material, and a preparation method thereof.

### BACKGROUND ART

Molecular imaging collectively refers to a number of techniques that enable researchers to observe genes, proteins, and other molecules performing a variety of functions in the body, and they have rapidly progressed, thanks to advances in cell biology, biochemical materials, and computer analysis. Unlike X-ray, ultrasound, and other conventional techniques which give doctors only such anatomical clues as the size of a tumor, molecular imaging could help track the underlying causes of disease, because it shows the motion of tumors at the molecular level. It has been reported that such molecular imaging techniques will substitute for breast X-ray examination, tissue biopsy and other medical examinations in the future.

As imaging agents in such molecular imaging techniques, semiconductor nanoparticles that absorb and emit light in the visible light region have been used, but in recent years, molecular imaging studies employing NIR Q-dots (semiconductor nanoparticles that absorb and emit light in the near-infrared region (800-2000 nm)), the intracellular permeability of which is about 10-fold higher than that of said semiconductor nanoparticles, have been actively conducted.

Meanwhile, Korean Patent Registration 541282 discloses a technique capable of specifically recognizing liver cells using superparamagnetic iron oxide nanoparticles as liver contrast agents. Also, U.S. Pat. No. 6,754,521 discloses techniques of recognizing veins and arteries using magnetic resonance imaging. Furthermore, WO 2006/067679 discloses a technique relating to a magnetic resonance imaging system which uses magnetic nanoparticles.

As described above, various attempts to apply magnetic particles in the medical and pharmaceutical fields have been continuously made, and study results resulting therefrom have been applied in industrial practice. However, such magnetic particles are not easy to apply to in vitro studies such as cell studies, because an external strong magnetic field is required to use magnetic properties. Accordingly, it is urgent to develop technology relating to particles having not only magnetic properties, but also fluorescent properties which can be observed and measured in vitro. However, the results of studies on the development of such technology are still insufficient.

Recently, a patent application relating to magnetic nanoparticles, which have optical properties and magnetic properties simultaneously and are covered with a silica shell, was filed (Korean Patent Publication 2007-0029030). However, in said patent application, fluorescent materials exhibiting optical properties are limited to organic fluorescent materials, and magnetic nanoparticles, which can be finally obtained, are also limited to water-soluble magnetic nanoparticles. In addition, there is a technical problem in that the silica shell must be surface-modified in a separate process in order to apply the nanoparticles to the human body.

Accordingly, the present inventors have made many efforts to solve the above-described problems occurring in the prior art and, as a result, have prepared biocompatible polymer particles, containing a magnetic nanomaterial and an NIR (near infrared) fluorescent material, using a double emulsion process or an emulsion solvent evaporation process, and have found that the prepared polymer particles show magnetic properties and fluorescent properties simultaneously, and thus are suitable for use in NIR/MR bimodal molecular imaging, thereby completing the present invention.

### SUMMARY OF INVENTION

It is an object of the present invention to provide a method for preparing polymer particles showing magnetic properties and fluorescent properties simultaneously.

Another object of the present invention is to provide polymer particles for NIR/MR bimodal molecular imaging, containing a magnetic nanomaterial and an NIR fluorescent material, and a contrast agent for NIR/MR bimodal molecular imaging containing said polymer particles.

To achieve the above objects, in one aspect, the present invention provides a method for preparing polymer particles showing magnetic properties and fluorescent properties simultaneously, the method comprising the steps of: (a) preparing a first dispersion (W₁/O) by dispersing an aqueous solution (W₁), containing a magnetic nanomaterial and an NIR fluorescent material dissolved therein, in an organic polymer solution (O), wherein any one or more of the magnetic nanomaterial and the NIR fluorescent material are hydrophilic; (b) preparing a second dispersion (W₁/O/W₂) by dispersing the first dispersion in an aqueous emulsifier solution (W₂); and (c) removing the organic solvent of the organic polymer solution of the step (a) and the aqueous emulsifier solution of the step (b) by stirring and centrifuging the second dispersion, and then collecting polymer particles.

In another aspect, the present invention provides a method for preparing polymer particles showing magnetic properties and fluorescent properties simultaneously, the method comprising the steps of: (a) preparing a mixed solution (O) by mixing an organic solution of hydrophobic magnetic nanomaterial with an organic solution of hydrophobic NIR fluorescent material, and then adding and dissolving a polymer in the mixture; (b) preparing a dispersion (O/W) by dispersing the mixed solution in an aqueous emulsifier solution (W); and (c) removing the organic solvent of the organic solution of the step (a) and the aqueous emulsifier solution of the step (b) by stirring and centrifuging the dispersion, and then collecting polymer particles.

In the present invention, the dispersion of the step (b) is preferably carried out using ultrasonic waves, and the polymer particles of the step (c) are preferably nanoparticles. In this case, the polymer nanoparticles have a particle diameter of 50-1000 nm.

In still another aspect, the present invention provides polymer nanoparticles for near infrared (NIR)/magnetic resonance (MR) bimodal molecular imaging, which are prepared according to any one of said methods, contain a magnetic nanomaterial and an NIR fluorescent material, and have a particle diameter of 50-1000 nm. Also, the present invention provides a contrast agent for NIR/MR bimodal molecular imaging, which contains said polymer nanoparticles.

In the present invention, the dispersion of the step (b) is preferably carried out using a homogenizer, and the polymer particles of the step (c) are preferably microparticles. In this case, the polymer microparticles have a particle diameter of 0.1-100 μm.

In still another aspect, the present invention provides polymer microparticles for near infrared (NIR)/magnetic resonance (MR) bimodal molecular imaging, which are prepared according to any one of said methods, contain a magnetic nanomaterial and an NIR fluorescent material, and have a particle diameter of 0.1-100 μm. Also, the present invention provides a contrast agent for NIR/MR bimodal molecular imaging, which contains said polymer microparticles.

Other features and aspects of the present invention will be apparent from the following detailed description and the appended claims.

### BRIEF DESCRIPTION OF DRAWINGS

FIG. 1 shows a transmission electron micrograph of polymer particles for NIR/MR bimodal molecular imaging.

FIG. 2 shows scanning electron micrographs of polymer nanoparticles and microparticles for NIR/MR bimodal molecular polymer imaging.

FIG. 3 is an MR image of a PLGA/magnetite/ICG nanoparticle solution.

FIG. 4 is an NIR image of a PLGA/magnetite/ICG nanoparticle solution.

FIG. 5 illustrates photographs showing the magnetic properties (b) and fluorescent properties (c) of a PLGA/magnetite/quantum dot nanoparticle solution (a).

### DETAILED DESCRIPTION OF THE INVENTION, AND PREFERRED EMBODIMENTS

In the present invention, biocompatible polymer particles showing NIR fluorescent properties and magnetic properties simultaneously are prepared using a double-emulsion method or an emulsion solvent evaporation method.

In the present invention, the double-emulsion method uses W₁/O/W₂ type emulsions (water-in-oil-in water). Specifically, the double-emulsion method refers to a method of impregnating a water-soluble material into oil-phase polymer particles dispersed in an aqueous solution (Cohen, S. et al., Pharm. Res., 8:713, 1991).

In the present invention, according to the double-emulsion method, polymer particles for NIR/MR bimodal molecular imaging are prepared by dispersing a mixed aqueous solution (W₁) of a magnetic nanomaterial and an NIR fluorescent material in an organic polymer solution (O), and then dispersing the organic polymer solution, containing the mixed aqueous solution dispersed therein, in an aqueous emulsifier solution (W₂), wherein any one of the magnetic material and the NIR fluorescent material is hydrophilic.

In one aspect, the present invention relates to a method for preparing polymer particles showing magnetic properties and fluorescent properties simultaneously, the method comprising the steps of: (a) preparing a first dispersion (W₁/O) by dispersing an aqueous solution (W₁), containing a magnetic nanomaterial and an NIR fluorescent material dissolved therein, in an organic polymer solution (O), wherein any one or more of the magnetic nanomaterial and the NIR fluorescent material are hydrophilic; (b) preparing a second dispersion (W₁/O/W₂) by dispersing the first dispersion in an aqueous emulsifier solution (W₂); and (c) removing the organic solvent of the organic polymer solution of the step (a) and the aqueous emulsifier solution of the step (b) by stirring and centrifuging the second dispersion, and then collecting polymer particles.

In the present invention, the organic solvent of the organic polymer solution in the step (a) is preferably one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

In the present invention, as an emulsion stabilizer, a protein having affinity for said magnetic nanomaterial or NIR fluorescent material may additionally be dissolved in the mixed aqueous solution of a magnetic material or an NIR fluorescent material.

In the present invention, the protein having affinity for said magnetic nanomaterial or NIR fluorescent material is preferably selected from the group consisting of serum albumin, serum globulin, serum fibrinogen, lipoprotein and transferrin. Herein, examples of the serum protein include albumin, globulin, fibrinogen, etc. In general, serum albumin has a wide range of functions, including nutrition by non-covalent binding, the regulation of osmotic pressure in the human body, the delivery of calcium ions, various metal ions, low molecular materials, bilirubin, drugs, and steroids, etc. In addition, because serum albumin has a function of binding such endogenous and exogenous materials, it can be used for the treatment of diseases, such as chronic renal failure, liver cirrhosis and shock disorders, and hypovolemia (Gayathri, V. P., Drug Development Research, 58:219, 2003).

Meanwhile, in the present invention, if both magnetic nanomaterial and NIR fluorescent material are hydrophobic, polymer particles for NIR/MR bimodal molecular imaging can be prepared using an emulsion solvent evaporation method.

In the present invention, the emulsion solvent evaporation method uses an O/W type emulsion (oil in water). Specifically, the emulsion solvent evaporation method is a method of obtaining polymer particles by dispersing an oil-phase material in an aqueous solution and removing an oil-phase organic solvent from the solution by evaporation (Kawashima et al., J. Pharm. Sci., 78:68, 1989).

In the present invention, the dispersion of a hydrophobic magnetic nanomaterial and hydrophobic NIR fluorescent material in the organic polymer solvent is preferably carried out by emulsion solvent evaporation (oil-in-water). Herein, the term “emulsion solvent evaporation” refers to a state in which an oil phase is dispersed in an aqueous phase while forming droplets, and then the solvent spontaneously evaporates with the passage of time. In the present invention, the mixed solution of a hydrophobic magnetic nanomaterial and a hydrophobic NIR fluorescent material is dispersed together with the organic polymer solution.

In another aspect, the present invention provides a method for preparing polymer particles showing magnetic properties and fluorescent properties simultaneously, the method comprising the steps of: (a) preparing a mixed solution (O) by mixing an organic solution of hydrophobic magnetic nanomaterial with an organic solution of hydrophobic NIR fluorescent material, and then adding and dissolving a polymer in the mixture; (b) preparing a dispersion (O/W) by dispersing the mixed solution in an aqueous emulsifier solution (W); and (c) removing the organic solvent of the organic solution of the step (a) and the aqueous emulsifier solution of the step (b) by stirring and centrifuging the dispersion, and then collecting polymer particles.

In the present invention, the organic solvent of the organic solution of hydrophobic magnetic nanomaterial in the step (a) is preferably one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene. The organic solvent of the organic solution of hydrophobic NIR fluorescent material in the step (a) is preferably one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

In the present invention, the polymer is preferably a biodegradable polyester polymer. The biodegradable polyester polymer is preferably selected from the group consisting of poly-L-lactic acid, poly-glycol acid, poly-D-lactic acid-co-glycol acid, poly-L-lactic acid-co-glycol acid, poly-D,L-lactic acid-co-glycol acid, poly-caprolactone, poly-valerolacton, poly-hydroxy butyrate and poly-hydroxy valerate.

As the biodegradable polyester polymer in the present invention, poly-L-lactic acid-co-glycolic acid) (PLGA) is preferably used. PLGA is a material approved for medical use by the US FDA, and it has no toxicity problem, and thus can be easily used for medical applications, such as drug delivery carriers and biomaterials, compared to other polymers.

In the present invention, the magnetic nanomaterial is preferably selected from the group consisting of Fe, Mn, Co, Gd, praseodymium (PR), samarium (Sm), eupium (Eu), terbium (Tb), dysprosium (Dy), holmium (Ho), erbium (Er), thulium (Tm), ytterbium (Yb) and lutetium (Lu).

As the magnetic nanomaterial in the present invention, iron oxide nanoparticles are preferably used. The magnetic nanoparticles allows the polymer particles prepared according to the present invention to show magnetic properties, thus making it possible to carry out magnetic resonance observation using the polymer particles.

In particular, the above-listed magnetic nanomaterials may be divided, according to the preparation method, into hydrophilic magnetic nanomaterials and hydrophobic magnetic nanomaterials. For example, in the preparation of metallic magnetic nanomaterials, the reduction of metal salt can be used to prepare hydrophilic magnetic nanomaterials, and the high temperature thermal decomposition of metal salt can be used to prepare hydrophobic magnetic nanomaterials. However, it is obvious to those skilled in the art that the surface properties of the magnetic nanomaterials prepared according to any method of the above-described methods can be changed from hydrophilic properties to hydrophilic properties or vice versa using an amphiphilic emulsifier and polymer and that the physical properties of the same magnetic nanomaterials can be changed to hydrophilic or hydrophobic properties according to the above-mentioned methods (Taeghwan Hyeon et al., Chem. Commun., 927, 2003; Hironori Iida et al., J. Colloid Interface Sci., 314:274, 2007). Accordingly, a magnetic nanomaterial showing hydrophilic properties or hydrophobic properties according to the preparation method as described above can be selected and applied in the present invention.

In the present invention, the NIR fluorescent material is preferably either an inorganic material selected from the group consisting of CdSe, CdSe/ZnS, CdTe/CdS, CdTe/CdTe, ZnSe/ZnS, ZnTe/ZnSe, PbSe, PbS, InAs, InP, InGaP, InGaP/ZnS and HgTe or an organic material selected from the group consisting of Cy3.5, Cy5, Cy5.5, Cy7, ICG(Indocyanine Green), Cypate, ITCC, NIR820, NIR2, IRDye78, IRDye80, IRDye82, Oxazines such as Cresyl Violet, Nile Blue, Oxazine 750, Rhodamines such as Rhodamine800, and Texas Red.

As the NIR fluorescent material in the present invention, a quantum dot or indocyanine green (ICG) is preferably used. The NIR fluorescent material allows the polymer particles prepared according to the present invention to show NIR fluorescent properties, thus making it possible to perform fluorescent image observation using the polymer particles.

In particular, NIR fluorescent materials including organic materials and inorganic materials can be hydrophilic or hydrophobic depending on the preparation method. NIR metal quantum dots, which are typical examples of inorganic NIR fluorescent materials, show hydrophobic properties, when they are prepared according to a wet chemical method of growing metal salts into nanocrystals at high temperature. On the other hand, they show hydrophilic properties, when the surface thereof are subjected to phase transition using a compound that binds specifically to the surface of the NIR metal quantum dots. In addition, the ICG, which is a typical example of organic NIR fluorescent material, becomes hydrophilic or hydrophobic depending on the molecular structure, when it is synthesized by an organic chemical synthesis method (Chiu-Ting Cheng et al., J. Materials Chemistry, 15:3409; Yuhui Lin et al., Bioconjugate Chem., 13:605). Accordingly, an NIR fluorescent material showing hydrophilic properties or hydrophobic properties depending on the preparation method as described above can be selected and applied in the present invention.

In the present invention, the emulsifier is preferably selected from the group consisting of PVA, non-ionic surfactants, cationic surfactants, anionic surfactants and amphoteric surfactants.

The aqueous emulsifier solution that is used in the present invention is prepared by dissolving an emulsifier in triple-distilled water. As the aqueous emulsifier solution in the present invention, a polyvinyl acetate (PVA) solution is preferably used. PVA functions as a surfactant for stabilizing polymer particles. Examples of emulsifiers which can be used in the present invention include, but are not limited to, in addition to PVA, polyalcohol derivatives, such as glycerin monostearate and stearin, non-ionic surfactants, including sorbitan esters and polysorbates, cationic surfactants such as cetyltrimethyl ammonium bromide, anionic surfactants, such as sodium lauryl sulfate, alkyl sulfonate and alkylaryl sulfonate, and amphoteric surfactants, such as higher alkylamino acid, polyamino monocarbonic acid and lecithin.

In the present invention, the dispersion of a magnetic nanomaterial and an NIR fluorescent material in the organic polymer solution is preferably a reverse emulsion (water-in-oil). Herein, the reverse emulsion refers to a form in which an aqueous phase is dispersed in an oil phase while forming droplets. In the present invention, the reverse emulsion indicates a form in which a mixed aqueous solution of protein, magnetic nanomaterial and NIR fluorescent material is dispersed in the organic polymer solution while forming droplets.

In the present invention, when the organic polymer solution containing the mixed aqueous solution of a magnetic nanomaterial and an NIR fluorescent material dispersed therein is dispersed in the aqueous emulsifier solution using ultrasonic waves or a homogenizer, the aqueous emulsifier solution forms droplets. At this time, polymer particles can be obtained by removing the organic solvent of the organic polymer solution and then solidifying the remaining polymer.

In the present invention, the finally obtained polymer particles may be nano- or microparticles, and this difference in particle size is attributable to the difference in the degree of dispersion by mechanical stirring means such as ultrasonic waves or a homogenizer used to disperse the organic polymer solution (containing the mixed aqueous solution of a magnetic nanomaterial and an NIR fluorescent material dispersed therein) in the aqueous emulsifier solution. Specifically, ultrasonic waves employ strong wavelengths, and thus disperse the mixed polymer solution more finely through strong vibration, such that the mixed aqueous solution of protein, magnetic nanomaterial and NIR fluorescent material is more strongly dispersed into the aqueous emulsifier solution, thus obtaining polymer nanoparticles. On the other hand, the degree of dispersion of a solution by mechanical stirring means such as a homogenizer varies depending on stirring speed (revolution per minute (R.P.M.)), and the homogenizer disperses the mixed aqueous solution of protein, magnetic nanomaterial and NIR fluorescent material into the aqueous emulsifier solution by mechanical stirring at a relatively low stirring speed compared to that of ultrasonic waves, thus obtaining polymer microparticles.

Specifically, if the dispersion of the step (b) is carried out using ultrasonic waves, polymer nanoparticles having a particle diameter of 50-1000 nm are obtained. On the other hand, if the dispersion of the step (b) is carried out using a homogenizer, polymer microparticles having a particle diameter of 0.1-100 μm are obtained.

In still another aspect, the present invention relates to polymer nanoparticles for near infrared (NIR)/magnetic resonance (MR) bimodal molecular imaging, which contain a magnetic nanomaterial and an NIR fluorescent material, and have a particle diameter of 50-1000 nm. Also, the present invention relates to a contrast agent for NIR/MR bimodal molecular imaging, which contains said polymer nanoparticles.

In still another aspect, the present invention relates to polymer microparticles for near infrared (NIR)/magnetic resonance (MR) bimodal molecular imaging, which contain a magnetic nanomaterial and an NIR fluorescent material, and have a particle diameter of 0.1-100 μm. Also, the present invention relates to a contrast agent for NIR/MR bimodal molecular imaging, which contains said polymer microparticles.

The polymer particles prepared according to the present invention are multifunctional polymer particles, which contain a magnetic material and an NIR fluorescent material and thus show magnetic properties and NIR fluorescent properties simultaneously. Thus, the polymer particles can be used as contrast agents for NIR/MR bimodal molecular imaging.

### EXAMPLES

Hereinafter, the present invention will be described in further detail with reference to examples. It is to be understood, however, that these examples are for illustrative purposes only and are not to be construed to limit the scope of the present invention.

### Example 1

### Preparation of PLGA/magnetite/ICG nanoparticles

100 mg of PLGA was dissolved in 2 ml of methylene chloride to prepare an organic solution of PLGA, and 15 mg of HSA (human serum albumin), 100 μl of hydrophilic magnetite (5 mg/mi) and 5 mg of hydrophilic indocyanine green (ICG) were sequentially dissolved in 2504 of triple-distilled water to prepare a mixed aqueous solution. The mixed aqueous solution was dissolved and stirred in the PLGA organic solution to prepare a first dispersion. The first dispersion was slowly added dropwise to 30 ml of 4%-PVA solution, while it was dispersed using a probe-type ultrasonic processor (700 W and 20 kHz) at an output of 95% for 5 minutes and stirred overnight in order to remove the organic solvent, thus preparing a second dispersion. In order to remove the remaining PVA, the second dispersion was centrifuged at 18000 rpm for 20 minutes, the supernatant was decanted, distilled water was added to the remaining material, and the dispersion was re-dispersed using ultrasonic waves and then centrifuged again. This centrifugation, decantation, dissolution and re-dispersion process was repeated three times. Then, the resulting PLGA/magnetite/ICG nanoparticles were freeze-dried and stored at 4° C.

The obtained PLGA/magnetite/ICG nanoparticles were observed with a scanning electron microscope (SEM), and as a result, the nanoparticles had a particle diameter of 50-1000 nm (FIG. 2(a)).

### Example 2

### Preparation of porous PLGA/magnetite/ICG Microparticles

100 mg of PLGA was dissolved in 2 ml of methylene chloride to prepare an organic solution of PLGA, and 15 mg of HSA (human serum albumin), 1004 of hydrophilic magnetite (5 mg/mi) and 5 mg of hydrophilic indocyanine green (ICG) were sequentially dissolved in 2504 of triple-distilled water to prepare a mixed aqueous solution. The mixed aqueous solution was dissolved and stirred in the PLGA organic solution to prepare a first dispersion. The first dispersion was slowly added dropwise to 30 ml of 4%-PVA solution, while it was dispersed using a homogenizer at 25000 rpm for 5 minutes and stirred overnight in order to remove the organic solvent, thus preparing a second dispersion. In order to remove the remaining PVA, the second dispersion was centrifuged at 8000 rpm for 20 minutes, the supernatant was decanted, distilled water was added to the remaining material, and the dispersion was re-dispersed using ultrasonic waves and then centrifuged again. This centrifugation, decantation, dissolution and re-dispersion process was repeated three times. Then, the resulting PLGA/magnetite/ICG microparticles were freeze-dried and stored at 4° C.

The obtained PLGA/magnetite/ICG microparticles were observed with a scanning electron microscope (SEM), and as a result, the porous microparticles had a particle diameter of 1-100 μm (FIG. 2(b)).

### Example 3

### Preparation of PLGA/Magnetite/ICG Nanoparticles

100 mg of PLGA was dissolved in 2 ml of methylene chloride to prepare an organic solution of PLGA, and 15 mg of HSA (human serum albumin) and 5 mg of hydrophilic indocyanine green (ICG) were sequentially dissolved in 250 μl of 28 mg/ml resovist (Sherring, Germany) to prepare a mixed aqueous solution. Herein, the resovist is an aqueous solution hydrophilic magenetic nanoparticles, which is used as a commercially contrast agent for MRI. The mixed aqueous solution was dissolved and stirred in the PLGA organic solution to prepare a first dispersion. The first dispersion was slowly added dropwise to 30 ml of 4%-PVA solution, while it was dispersed using a probe-type ultrasonic processor (700 W and 20 kHz) at an output of 95% for 5 minutes and stirred overnight in order to remove the organic solvent, thus preparing a second dispersion. In order to remove the remaining PVA, the second dispersion was centrifuged at 18000 rpm for 10 minutes, the supernatant was decanted, distilled water was added to the remaining material, and the dispersion was re-dispersed using ultrasonic waves and then centrifuged again. This centrifugation, decantation, dissolution and re-dispersion process was repeated three times. Then, the resulting PLGA/magnetite/ICG nanoparticles were freeze-dried and stored at 4° C.

The obtained PLGA/magnetite/ICG nanoparticles were observed with a scanning electron microscope (SEM), and as a result, the nanoparticles had a particle diameter of 50-1000 nm (FIG. 2(c)).

### Example 4

### Preparation of PLGA/magnetite/Quantum Dot Nanoparticles

An organic solution of hydrophobic magnetite dissolved in CHCl₃ solution was mixed with an organic solution of hydrophobic quantum dots dissolved in CHCl₃ solution, and then 100 mg of PLGA was added thereto, thus preparing a mixed solution. The mixed solution was slowly added dropwise to 30 ml of 4%-PVA solution, while it was dispersed using a probe-type ultrasonic processor (700 W and 20 kHz) at an output of 95% for 5 minutes and stirred overnight in order to remove the organic solvent, thus preparing a second dispersion. In order to remove the remaining PVA, the second dispersion was centrifuged at 18000 rpm for 10 minutes, the supernatant was decanted, distilled water was added to the remaining material, and the dispersion was re-dispersed using ultrasonic waves and then centrifuged again. This centrifugation, decantation, dissolution and re-dispersion process was repeated three times. Then, the resulting PLGA/magnetite/quantum dot nanoparticles were freeze-dried and stored at 4° C.

The obtained PLGA/magnetite/quantum dot nanoparticles were observed with a scanning electron microscope (SEM), and as a result, the nanoparticles had a particle diameter of 50-1000 nm (FIG. 2(d)).

### Experimental Example 1

### Measurement of Magnetic Properties and Fluorescent Properties of PLGA/Magnetite/ICG Nanoparticles

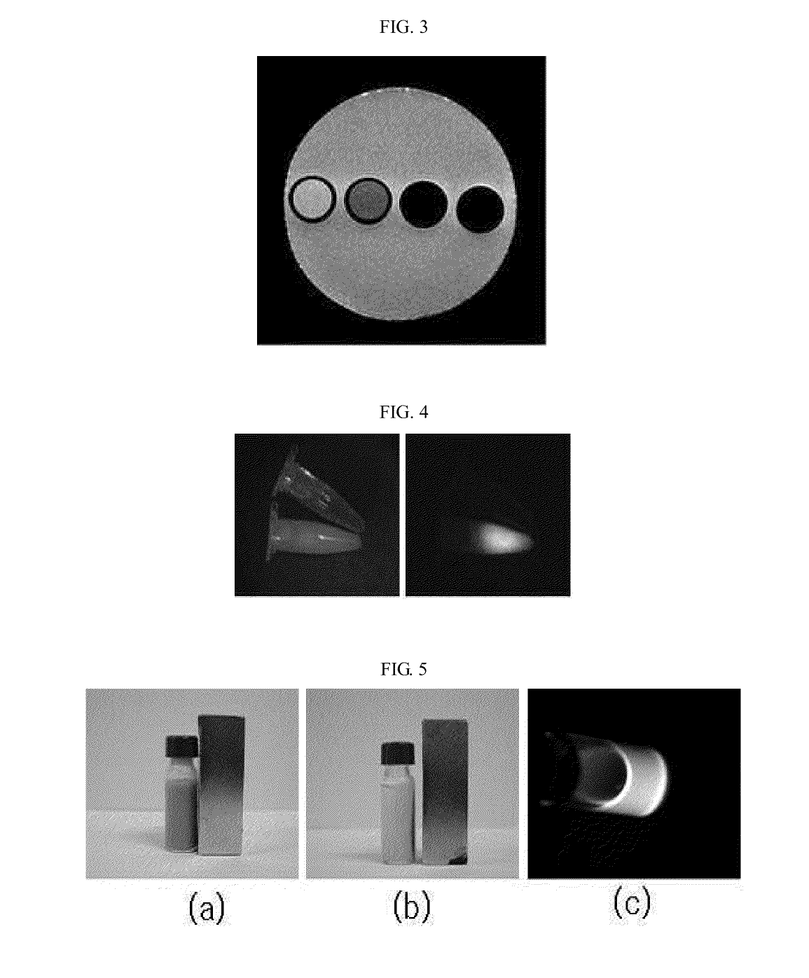

A magnetic resonance image of the PLGA/magnetite/ICG nanoparticles prepared in Example 1 was photographed and, as a result, it was observed that the nanoparticles showed magnetic properties (FIG. 3). In addition, the nanoparticles were photographed in the NIR region and, as a result, it was observed that the nanoparticles showed fluorescent properties (FIG. 4).

Accordingly, it was observed that the PLGA/magnetite/ICG nanoparticles prepared in Example 1 of the present invention showed magnetic properties and fluorescent properties simultaneously.

### Experimental Example 2

### Measurement of Magnetic Properties and Fluorescent Properties of PLGA/Magnetite/Quantum Dot Nanoparticles

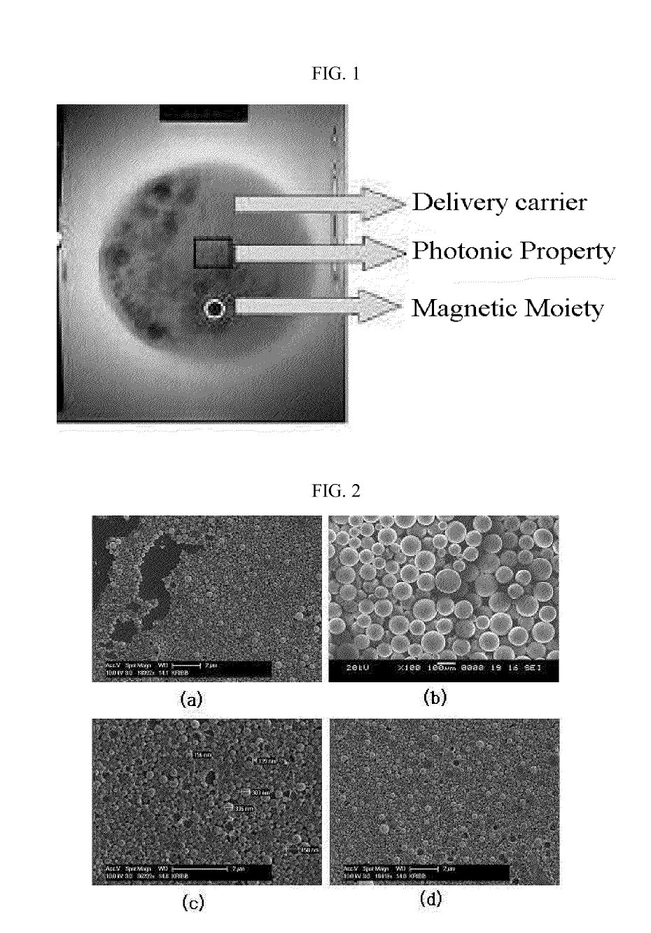

The PLGA/magnetite/quantum dot nanoparticles prepared in Example 4, containing magnetite nanoparticles and quantum dot nanoparticles, were observed with a transmission electron microscope (FIG. 1). Moreover, it was observed that, when a magnet was applied to the solution of the polymer nanoparticles (FIG. 5(a)), the PLGA/magnetite/quantum dot nanoparticles in the solution moved toward the magnet (FIG. 5(b)). In addition, it was observed that, when the nanoparticle solution was irradiated with a 365-nm UV or 760-nm Near-IR laser, the PLGA/magnetite/quantum dot nanoparticles in the solution showed NIR fluorescence at 800 nm (FIG. 5(c)).

Accordingly, it was confirmed that the PLGA/magnetite/ICG nanoparticles prepared in Example 4 of the present invention showed magnetic properties and fluorescent properties simultaneously.

### INDUSTRIAL APPLICABILITY

As described above, according to the present invention, nano- or micro-sized, multifunctional polymer particles showing magnetic properties and fluorescent properties simultaneously can be prepared. The polymer particles are useful as contrast agents for NIR/MR bimodal molecular imaging.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof

1. A method for preparing polymer particles showing magnetic properties and fluorescent properties simultaneously, the method comprising the steps of:
(a) preparing a first dispersion (W₁/O) by dispersing an aqueous solution (W₁), containing a magnetic nanomaterial and an NIR fluorescent material dissolved therein, in an organic polymer solution (O), wherein any one or more of the magnetic nanomaterial and the NIR fluorescent material are hydrophilic;
(b) preparing a second dispersion (W₁/O/W₂) by dispersing the first dispersion in an aqueous emulsifier solution (W₂); and
(c) removing the organic solvent of the organic polymer solution of the step (a) and the aqueous emulsifier solution of the step (b) by stirring and centrifuging the second dispersion, and then collecting polymer particles.

2. A method for preparing polymer particles showing magnetic properties and fluorescent properties simultaneously, the method comprising the steps of:
(a) preparing a mixed solution (O) by mixing an organic solution of hydrophobic magnetic nanomaterial with an organic solution of hydrophobic NIR fluorescent material, and then adding and dissolving a polymer in the mixture;
(b) preparing a dispersion (O/W) by dispersing the mixed solution in an aqueous emulsifier solution (W); and
(c) removing the organic solvent of the organic solution of the step (a) and the aqueous emulsifier solution of the step (b) by stirring and centrifuging the dispersion, and then collecting polymer particles.

3. The method for preparing polymer particles showing magnetic properties and fluorescent properties simultaneously according to claim 1, wherein the polymer is a biodegradable polyester polymer.

4. The method for preparing polymer particles showing magnetic properties and fluorescent properties simultaneously according to claim 3, wherein the biodegradable polyester polymer is selected from the group consisting of poly-L-lactic acid, poly-glycol acid, poly-D-lactic acid-co-glycol acid, poly-L-lactic acid-co-glycol acid, poly-D,L-lactic acid-co-glycol acid, poly-caprolactone, poly-valerolacton, poly-hydroxy butyrate and poly-hydroxy valerate.

5. The method for preparing polymer particles showing magnetic properties and fluorescent properties simultaneously according to claim 1, wherein the organic solvent of the organic polymer solution in the step (a) is one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

6. The method for preparing polymer particles showing magnetic properties and fluorescent properties simultaneously according to claim 1, wherein the magnetic nanomaterial is selected from the group consisting of Fe, Mn, Co, Gd, praseodymium (PR), samarium (Sm), eupium (Eu), terbium (Tb), dysprosium (Dy), holmium (Ho), erbium (Er), thulium (Tm), ytterbium (Yb) and lutetium (Lu).

7. The method for preparing polymer particles showing magnetic properties and fluorescent properties simultaneously according to claim 1, wherein the NIR fluorescent material is either an inorganic material selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe or an organic material selected from the group consisting of Cy3.5, Cy5, Cy5.5, Cy7, ICG(Indocyanine Green), Cypate, ITCC, NIR820, NIR2, IRDye78, IRDye80, IRDye82, Oxazines such as Cresyl Violet, Nile Blue, Oxazine 750, Rhodamines such as Rhodamine800, and Texas Red.

8. The method for preparing polymer particles showing magnetic properties and fluorescent properties simultaneously according to claim 1, wherein the emulsifier is selected from the group consisting of PVA, non-ionic surfactants, cationic surfactants, anionic surfactants and amphoteric surfactants.

9. The method for preparing polymer particles showing magnetic properties and fluorescent properties simultaneously according to claim 2, wherein the organic solvent of the organic solution of hydrophobic magnetic nanomaterial in step the (a) is one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

10. The method for preparing polymer particles showing magnetic properties and fluorescent properties simultaneously according to claim 2, wherein the organic solvent of the organic solution of hydrophobic NIR fluorescent material in the step (a) is preferably one or a mixture of two or more solvents selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methylethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

11. The method for preparing polymer particles showing magnetic properties and fluorescent properties simultaneously according to claim 1, wherein the dispersion of the step (b) is carried out using ultrasonic waves or a homogenizer.

12. The method for preparing polymer particles showing magnetic properties and fluorescent properties simultaneously according to claim 1, wherein the dispersion of the step (b) is carried out using ultrasonic waves, and the polymer particles of the step (c) are nanoparticles.

13. The method for preparing polymer particles showing magnetic properties and fluorescent properties simultaneously according to claim 1, wherein the dispersion of the step (b) is carried out using a homogenizer, and the polymer particles of the step (c) are microparticle.

14. The method for preparing polymer particles showing magnetic properties and fluorescent properties simultaneously according to claim 1, wherein a protein having affinity for said magnetic nanomaterial or NIR fluorescent material is additionally dissolved in the mixed aqueous solution (W₁) of a magnetic material or an NIR fluorescent material in the step (a), as an emulsion stabilizer.

15. The method for preparing polymer particles showing magnetic properties and fluorescent properties simultaneously according to claim 14, wherein the protein having affinity for said magnetic nanomaterial or NIR fluorescent material is selected from the group consisting of serum albumin, serum globulin, serum fibrinogen, lipoprotein and transferrin

16. Polymer nanoparticles for near infrared (NIR)/magnetic resonance (MR) bimodal molecular imaging, which are prepared by the method of claim 12, contain a magnetic nanomaterial and an NIR fluorescent material and have a particle diameter of 50-1000 nm.

17. Polymer microparticles for near infrared (NIR)/magnetic resonance (MR) bimodal molecular imaging, which are prepared by the method of claim 13, contain a magnetic nanomaterial and an NIR fluorescent material and have a particle diameter of 0.1-100 μm.

18. A contrast agent for NIR/MR bimodal molecular imaging, which contains the polymer nanoparticles of claim 16.

19. A contrast agent for NIR/MR bimodal molecular imaging, which contains the polymer microparticles of claim 17.
